(19) United States
(12) Patent Application Publication
(10) Pub. No.: US 2019/0169658 A1
(43) Pub. Date: Jun. 6, 2019

(54) BACILLUS AEROLACTICUS FOR PRODUCING L-LACTIC ACID OR ITS SALTS FROM VARIOUS CARBON SOURCES

(71) Applicants: PTT Global Chemical Public Company Limited, Bangkok (TH); Chulalongkorn University, Bangkok (TH)

(72) Inventors: Nuttha Thongchul, Bangkok (TH); Kentaro Kodama, Ibarakiken (JP); Paphawee Nantarajit, Rayong (TH); Techanan Wikanda, Bangkok (TH); Phatthanon Prasitchoke, Bangkok (TH)

(73) Assignees: PTT Global Chemical Public Company Limited, Bangkok (TH); Chulalongkorn University, Bangkok (TH)

(21) Appl. No.: 16/095,012

(22) PCT Filed: Apr. 22, 2016
(86) PCT No.: PCT/TH2016/000040

§ 371 Date: Oct. 19, 2018

(51) Int. Cl. C12P 7/56; C12N 1/20; C12R 1/07; C12N 9/26

(52) U.S. Cl. CPC C12P 7/56; C12N 1/20; C12Y 302/01001; C12N 9/2411; C12R 1/07

(57) ABSTRACT

The present invention relates to a novel Bacillus aerolacticus BC-001 species and a method for producing lactic acid or its salts using said bacteria, wherein the novel bacteria species can produce L-lactic acid or its salts with high optical purity from carbohydrate carbon sources, including oligosaccharide, disaccharide and monosaccharide, wherein said bacteria can grow well under an aerobic condition, tolerate to high temperature in a range of 45 to 60° C.

### SUMMARY OF THE INVENTION

This invention relates to a novel Bacillus aerolacticus BC-001 species and a method for producing lactic acid or its salts using said bacteria, wherein said novel bacteria is deposited to NITE Patent Microorganisms Depositary (NPMD), Japan, Accession Number NITE BP-01943. Said Bacillus aerolacticus can produce L-lactic acid or its salts from various carbon sources.

Said bacteria can grow well under an aerobic condition, be tolerant to high temperature at more than 45° C., and can produce L-lactic acid or its salts with high optical purity.

Moreover, the invention relates to a method for producing L-lactic acid or its salts, comprising the following steps:

(1) cultivating the Bacillus aerolacticus BC-001 species to obtain a seed culture; and

(2) fermenting the seed culture obtained from step (1) in a carbon source.

### FIELD OF THE INVENTION

Biotechnology relates to bacteria that can produce lactic acid.

### BACKGROUND OF THE INVENTION

It is well known that lactic acid has been widely used in plastic, food, pharmaceutical, and cosmetic industries.

Lactic acid is a chiral molecule which its polarization property results in a categorization of lactic acid to 3 isomers that are L-lactic acid, D-lactic acid, and racemic lactic acid. For plastic industry, L-lactic acid is widely used, especially for production of polyester such as polylactic acid or poly(lactic-co-glycolic acid). Polymer produced from lactic acid has its advantage that it is biodegradable and biocompatible. Said polymer can be used in many applications such as fiber in textile, film, packaging, catgut, and scaffold in medical field.

At present, there are several production processes of lactic acid, such as chemical synthesis and biotechnology. Biotechnology possesses several advantages including the utilization of renewable resources for microbial fermentation such as tapioca, corn, wheat, or sugarcane. Moreover, the microbial fermentation is capable of producing lactic acid with high optical purity.

Most of industrial lactic acid productions are the fermentation of sugar such as glucose, sucrose, maltose, or other carbohydrates such as starch or cellulose, wherein microorganisms that can produce lactic acid are bacteria and fungi.

Bacteria in genus Lactobacillus, Leuconostoc, and Streptococcus are well known in the production of lactic acid from sugar under anaerobic condition, leading to low energy consumption and providing product with higher titer than from fungi. However, said bacteria group is fastidious bacteria which need vitamin and essential amino acids on its growth. Moreover, said bacteria group cannot produce enzyme to convert starch into sugar, leading to the need of pretreatment step prior to fermentation, which increases production cost.

Persson et al., (Biotechnol. Bioeng., 2001, 72, 269-277) and Soccol et al., (Biochem. Eng. J., 2003, 13, 205-218) disclosed the use of Lactobacilli bacteria in lactic acid production. However, said bacteria yielded the mixture of L- and D-lactic acids, thus additional purification process is required to separate such isomers before using them in the polylactic production, which was the limitation. Min Young Jung et al., (IJSEM, 2009, 59, 2226-2231) reported the new species of Bacillus that can produce lactic acid and named said species as Bacillus acidiproducens. However, as disclosed, Bacillus acidiproducens could not produce lactic acid from some carbon sources such as starch, and the optical purity of produced lactic acid was not discussed. To date, the efficiency of lactic acid production from said Bacillus acidiproducens has not been studied and reported yet.

One problem of polymer production from lactic acid is its high production cost. It is necessary to develop the lactic acid production process with lower production cost, increase yield and provide high optical purity isomer product. Therefore, there are several attempts to study and develop robust microorganisms that have ability to grow and proliferate with high rate, and can utilize low cost carbon sources as raw materials in lactic acid production.

One attempt to reduce lactic acid production cost is to use complex carbon sources derived from plant biomass, agricultural residues and industrial waste rather than an expensive monosaccharide in fermentation. However, most of lactic acid bacteria naturally found could not digest and utilize complex carbon sources. Thus, a pretreatment process is required prior to lactic acid fermentation. Several pretreatments can be used including mechanical treatment, heat treatment, chemical treatment, or enzyme treatment, depending on the physical, chemical, and nutritional properties of such carbon sources. Said pretreatment processes are generally performed at high temperature in a range from about 50 to about 60° C. Generally, bacteria cannot grow at said temperature, additional step is therefore needed prior to fermentation so as to reduce the temperature to a room temperature, resulting in a complexity of production process and increasing of production cost.

From the reasons mentioned above, there is a need for microorganisms that tolerate to high temperature, provide high lactic acid yield with high optical purity, and can utilize various carbon sources.

### BRIEF DESCRIPTION OF THE DRAWINGS

FIG. 1 shows nucleotide sequence of 16S rRNA gene of Bacillus aerolacticus BC-001.

FIG. 2 shows phylogenetic tree of Bacillus aerolacticus BC-001. Horizontal solid line shows differences in phylogenetic of BC-001 compared to the closest related type strain, Bacillus acidiproducens SL213^{T} strain (from IJSEM, 2009, 59, 2226-2231).

FIG. 3 shows micrograph from SEM of Bacillus aerolacticus BC-001 obtained from cultivation for 3 hours at temperature of 50° C., and at shaking speed of 250 rpm.

FIG. 4 shows optical density of Bacillus aerolacticus BC-001 at various initial concentrations of BC-001 and different cultivation periods.

### DETAILED DESCRIPTION OF THE INVENTION

### Definition

Technical terms or scientific terms used herein, have definitions as understood by those having an ordinary skill in the art, unless stated otherwise.

Equipment, apparatus, methods, or chemicals mentioned here means equipment, apparatus, methods or chemicals commonly operated or used by those skilled in the art, unless explicitly stated otherwise that they are equipment, apparatus, methods, or chemicals specifically used in this invention.

The use of the singular or plural nouns with the term “comprising” in the claims or in the specification refers to “one” and also “one or more”, “at least one”, and “one or more than one”.

Throughout this application, the term “about” is used to indicate that any value presented herein may potentially vary or deviate. Such variation or deviation may result from errors of apparatus, methods used in calculation or from individual operator implementing apparatus or methods. These include variations or deviations caused by the changes of physical properties.

“Starch” means purified starch, raw starch, liquefied starch, or any material that comprises starch or liquefied starch. Example of starch in this invention includes but not limited to tapioca starch, corn starch, wheat starch, or potato starch.

“Liquefied starch” means starch that is obtained from liquefaction process. Said process includes but not limited to the breakdown of starch structure by using physical method and/or chemical method such as heating, heating under pressure, chemical and enzyme treatments.

“Microaerobic condition” means condition that air has been controlled to be limited without adding additional air during fermentation or growth of the microorganisms.

Hereafter, invention embodiments are shown without any purpose to limit any scope of the invention.

The present invention relates to a novel thermotolerant Bacillus aerolacticus BC-001 species that can produce lactic acid from various carbon sources, and a method for producing L-lactic acid using said bacteria.

Bacillus aerolacticus BC-001 of this invention can grow well under an aerobic condition, be tolerant to high temperature at more than 45° C., and can produce L-lactic acid or its salts with high optical purity.

Bacillus aerolacticus BC-001 of this invention is deposited to NITE Patent Microorganisms Depositary (NPMD), Japan, under Budapest Treaty. The accession number given by NPMD is NITE BP-01943.

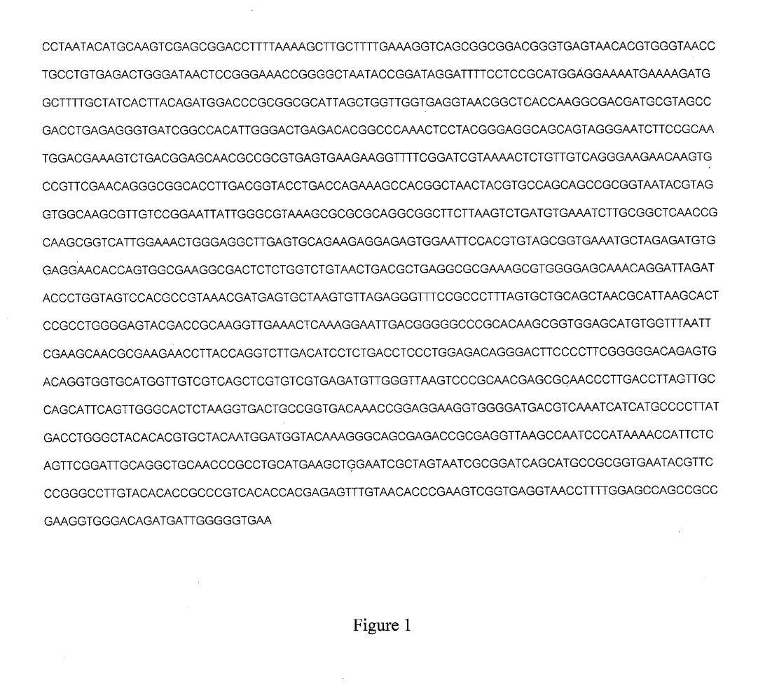

Bacillus aerolacticus BC-001 is a gram-positive bacteria with the nucleotide sequence of 16S rRNA as shown in FIG. 1.

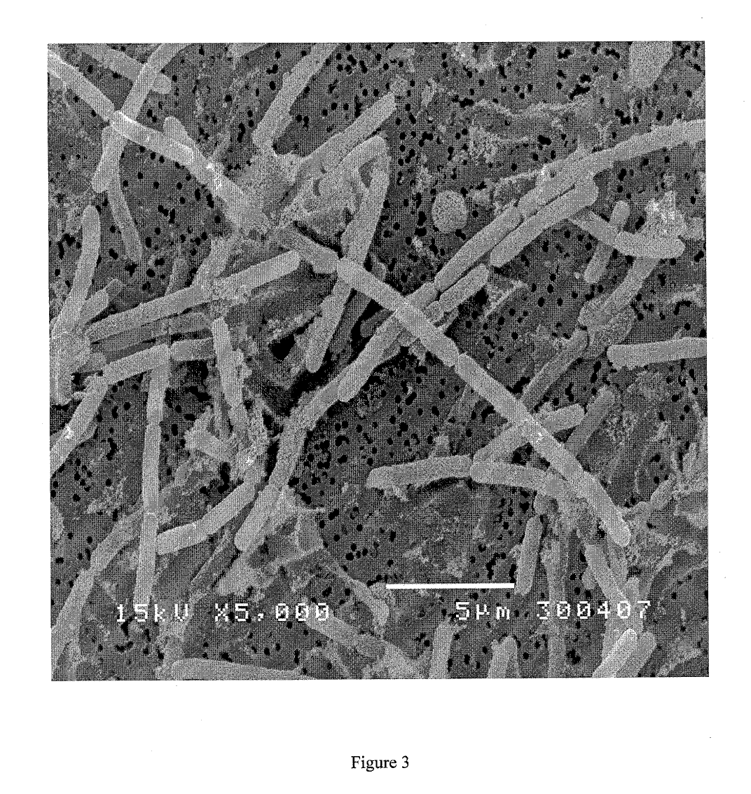

Bacillus aerolacticus BC-001 has the morphology as shown in FIG. 3.

Bacillus aerolacticus BC-001 is isolated from leaf and bark of tamarind in Lopburi, Thailand.

In one embodiment, said Bacillus aerolacticus BC-001 can grow well under an aerobic condition, be tolerant to a temperature in a range of around 45 to 60° C. Preferably, said Bacillus aerolacticus BC-001 can grow well under the aerobic condition, be tolerant to the temperature at around 50° C.

Bacillus aerolacticus BC-001 can produce L-lactic acid and its salts with optical purity more than 95%, preferably more than 99%.

In another embodiment, this invention relates to the method for producing L-lactic acid or its salts using Bacillus aerolacticus as described above.

The method for producing L-lactic acid or its salts, comprising the following steps:

(1) cultivating the Bacillus aerolacticus BC-001 species to obtain a seed culture; and

(2) fermenting the seed culture obtained from step (1) in a carbon source.

In one embodiment, the cultivation in step (1) may be performed for a period of about 2 to 10 hours, preferably from about 3 to 5 hours and most preferably about 5 hours. In one embodiment, a concentration of Bacillus aerolacticus species in the cultivation step is about 0.5 to 5% by volume, preferably about 0.5 to 2% by volume and most preferably about 1% by volume.

In one embodiment, the fermentation of seed culture in step (2) may be performed at a temperature in a range of about 45 to 60° C., preferably at about 50° C. In one embodiment, the fermentation of seed culture in step (2) may be performed under a microaerobic condition.

Carbon sources for the fermentation may be selected from, but not limited to, fermentable sugar, starch, liquefied starch, or a mixture thereof.

Fermentable sugar is any sugar that can be found in nature or any sugar derived from a substance comprising sugar. Said sugar may be modified or unmodified.

In one embodiment, the fermentable sugar is monosaccharide that may be selected from glucose, fructose, galactose, or a mixture thereof.

In one embodiment, the fermentable sugar is disaccharide that may be selected from sucrose, lactose, maltose, cellobiose, or a mixture thereof.

In one embodiment, the fermentable sugar is trisaccharide that may be selected from raffinose, isomaltotriose, maltotriose, nigerotriose, kestose, or a mixture thereof.

Preferably, the fermentable sugar is selected from glucose, sucrose, or a mixture thereof.

In one embodiment, starch is selected from tapioca starch, corn starch, wheat starch, potato starch, or a mixture thereof.

The liquefied starch is starch that is contacted with amylase enzyme.

More preferably, the concentration of carbon source in the fermentation of seed culture is in a range of about 50 to 200 g/L.

In one embodiment, the fermentation of seed culture in step (2) may further comprise the step of adding glucoamylase enzyme during the fermentation of seed culture. The following is property tests according to the invention, wherein the methods and equipment used in the tests are commonly used and are not intended to limit the scope of the invention.

Glucose, lactic acid, and by-product are analyzed by high performance liquid chromatography using a Shimadzu equipped with a Biorad, Aminex HPX-87H ion exclusion organic acid 300 mm×7.8 mm column, at a temperature around 45° C., and reflective index detector Shimadzu-RID-10A for detecting a signal comparing to a standard signal.

Optical purity of L-lactic acid is analyzed by a chiral column Sumipack Sumichiral OA5000 at a temperature of 40° C. Copper sulfate (CuSO₄) is used as an eluent with a flow rate of about 1 ml/min. The signals are detected by using a UV Detector at a wavelength of 254 nm.

Optical density (OD) of BC-001 during the cultivation or fermentation is analyzed by Spectrophotometry at a wavelength of 600 nm.

Yield is calculated from a ratio of an amount of produced lactic acid to an amount of carbon sources used during fermentation.

The following examples are presented to illustrate the present invention without limiting the scope of the invention.

Isolation and Characterization of Bacteria According to the Invention

Bacillus aerolacticus BC-001 is isolated from leaf and bark of tamarind, Lopburi, Thailand. A soil sample is added into a test tube filled with a medium for microorganism isolation, wherein said medium contains glucose in a concentration of around 10 to 15 g/L. The isolation is performed at the temperature of around 50° C. The colonies that can acidify the medium or give a clear zone are picked up. After that, a catalase test of obtained colonies is conducted to select colonies that can grow under an aerobic condition. After said method, Bacillus aerolacticus BC-001 that can produce lactic acid, grow under an aerobic condition and be tolerant to a high temperature is isolated from other bacteria strains.

Bacillus aerolacticus BC-001 isolated from above method is then analyzed for a nucleotide sequence of 16s rRNA, a phylogenetic tree, bacterial species identification by DNA-DNA hybridization, and a morphology. Results are showed in FIG. 1, 2, Table 1 and FIG. 3 respectively.

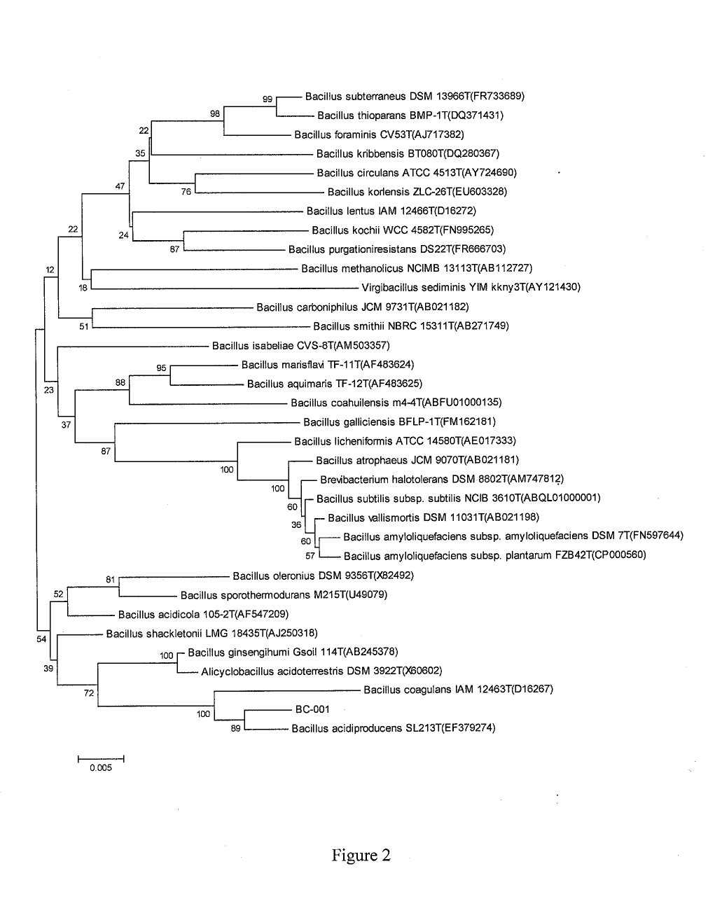

The phylogenetic tree in FIG. 2 shows that Bacillus aerolacticus BC-001 is closest to Bacillus acidiproducens SL213^{T} strain. The 16S rRNA gene sequence of BC-001 in FIG. 1 indicates 98.85% similarity to the SL213^{T} strain. Therefore, DNA-DNA hybridization of BC-001 is further conducted to determine the species of BC-001, wherein BC-001 and Bacillus acidiproducens 13078 type strains are used as DNA probes and Bacillus coagulans 6326 type strain is used as a negative control. The DNA-DNA hybridization result is shown in Table 1.

TABLE 1
DNA-DNA hybridization result of BC-001

| | | % |
|---|---|---|
| | | DNA-DNA |
| Isolate | DNA probe | hybridization |
| BC-001 | BC-001 | 100 |
| Bacillus acidiproducens | BC-001 | 58.7 |
| 13078 | | |
| Bacillus coagulans 6326 | BC-001 | 48.1 |
| BC-001 | Bacillus acidiproducens 13078 | 30.7 |
| Bacillus acidiproducens | Bacillus acidiproducens 13078 | 100 |
| 13078 | | |
| Bacillus coagulans 6326 | Bacillus acidiproducens 13078 | 34.7 |

From Table 1, when using BC-001 as DNA probe for Bacillus acidiproducens 13078 and Bacillus coagulans 6326 and using Bacillus acidiproducens 13078 as DNA probe for BC-001, % DNA-DNA hybridization is less than 70%. Therefore, it indicates that the BC-001 belongs to distinct species in Bacillus genus and is deposited at NITE Patent Microorganisms Depositary (NPMD), Japan, accession number NITE BP-01943 with the scientific name of Bacillus aerolacticus.

Concentration of BC-001 in Cultivation Step

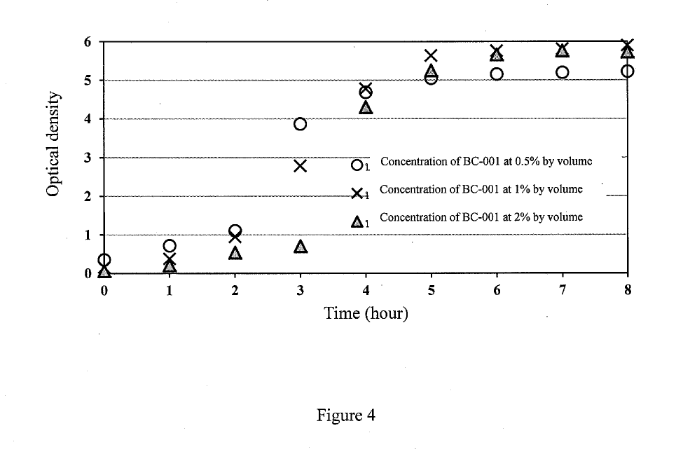

Bacillus aerolacticus BC-001 is added to the cultivation medium containing the following compositions per liter: about 10 g of glucose, about 15 g of yeast extract, about 4 g of ammonium chloride (NH₄Cl), about 5 g of calcium hydroxide, and about 20 ml of salt solution. Various concentrations of BC-001 are studied, including 0.5%, 1%, and 2% by volume. Thereafter, the mixture is shaken at around 250 rpm and the temperature about 50° C. The optical density (OD) of BC-001 is analyzed at different time. The result is shown in FIG. 4.

Study of Various Conditions in Cultivation and Fermentation Steps

To study the effect of cultivation period and aeration condition during fermentation on the ability to produce lactic acid of BC-001, various conditions in Table 1 are studied for lactic acid production.

The production of lactic acid is carried out by adding Bacillus aerolacticus BC-001 with a concentration of about 1% by volume in a culture medium containing the following composition per liter: about 10 g of glucose, about 15 g of yeast extract, about 4 g of ammonium chloride (NH₄Cl), about 5 g of calcium hydroxide and about 20 ml of salt solution. Thereafter, said mixture is shaken at around 250 rpm and a temperature of about 50° C. to obtain a seed culture. Then, about 25 ml of said seed culture is added into a flask filled with 25 ml of 200 g/L glucose solution by using calcium carbonate (Ca(CO)₃) to control pH to be about 6.5 to 6.8. After that, the fermentation of obtained seed culture is performed for 24 hours at the temperature about 50° C. with various aeration and shaking conditions. At the end of fermentation, the products are centrifuged at about 10,000 rpm for about 5 minutes. The obtained products are analyzed for the optical density of bacteria and amount of lactic acid produced. The results are shown in Table 2.

From Table 2, it is found that an increase of cultivation period, shaking speed during fermentation, and microaerobic condition during fermentation result in an increase of the optical density of bacteria, final concentration of lactic acid and the productivity of lactic acid. Therefore, according to above results, it can be summarized that the microareobic condition and shaking during fermentation of Bacillus aerolacticus can enhance the efficiency of lactic acid production.

TABLE 2
Lactic acid production of Bacillus aerolacticus BC-001 at various cultivation and fermentation conditions

| Cultivation step | | | Fermentation step | | | | |
|---|---|---|---|---|---|---|---|
| | Initial optical | Final optical | | Final optical | Final lactic acid | | |
| Time | density | density | | density | concentration | Yield | Productivity |
| (hour) | (OD) | (OD) | Shaking:Aeration | (OD) | (g/L) | (g/g) | (g/L/hr) |
| 3 | 0.4 | 3.1 | wo:an | 13.2 | 53.7 | 1.13 | 2.2 |
| | | | wo:micro | 19.7 | 73.1 | 1.06 | 3.0 |
| | | | w:an | 13.2 | 59.3 | 1.11 | 2.5 |
| | | | w:micro | 22.6 | 92.4 | 0.93 | 3.9 |
| 4 | 0.4 | 4.1 | wo:an | 13.8 | 54.4 | 1.24 | 2.3 |
| | | | wo:micro | 21.3 | 75.1 | 1.06 | 3.1 |
| | | | w:an | 13.2 | 58.9 | 1.21 | 2.5 |
| | | | w:micro | 25.3 | 92.2 | 0.93 | 3.8 |
| 5 | 0.4 | 4.6 | wo:an | 13.8 | 51.2 | 1.22 | 2.1 |
| | | | wo:micro | 20.2 | 71.1 | 1.06 | 3.0 |
| | | | w:an | 15.0 | 67.5 | 1.10 | 2.8 |
| | | | w:micro | 24.6 | 95.5 | 0.96 | 4.0 |

Remarks:
“wo” refers to fermentation without shaking
“w” refers to fermentation with shaking at about 250 rpm
“an” refers to fermentation under an anaerobic condition by using a flask with T-type silicone stopper and placed in an AnaeroPack
“micro” refers to fermentation under a microaerobic condition by using a flak with C-type silicone stopper

Ability to Produce Lactic Acid of BC-001 from Various Carbon Sources

In order to demonstrate that Bacillus aerolacticus BC-001 is able to produce lactic acid from various carbon sources, the carbon sources including glucose, sucrose and liquefied tapioca starch, are used in the production of lactic acid of BC-001. These carbon sources are intended to be examples selected to illustrate carbon sources previously described and do not limit the scope of the invention.

The production of lactic acid could be carried out by adding Bacillus aerolacticus BC-001 with a concentration of about 1% by volume into a culture medium containing the following composition per liter: about 10 g of glucose, about 15 g of yeast extract, about 4 g of ammonium chloride (NH₄Cl), about 5 g of calcium hydroxide, and about 20 ml of salt solution. The mixture is shaken at around 250 rpm and a temperature about 50° C. for about 5 hours to obtain seed culture. Then, 25 ml of said seed culture is added into a flask filled with the following carbon sources.

### Example 1

Carbon source is 25 ml of glucose solution with a concentration of 240 g/L by using calcium carbonate (Ca(CO)₃) to control pH to be in a range of about 6.5 to 6.8. After that, the fermentation of obtained seed culture is performed for about 48 hours at about 50° C. under a microaerobic condition and shaking speed around 250 rpm.

### Example 2

Carbon source is 25 ml of glucose solution with an initial concentration of 200 g/L by using calcium carbonate (Ca(CO)₃) to control pH to be in a range of about 6.5 to 6.8. After that, the fermentation of obtained seed culture is performed for about 24 hours at about 50° C. under a microaerobic condition and shaking speed around 250 rpm. Then, the glucose solution is added to adjust the concentration to be 150 g/L. Fermentation is further carried out for about 24 hours.

### Example 3

Carbon source is 25 ml of sucrose solution with a concentration of 300 g/L by using calcium carbonate (Ca(CO)₃) to control pH to be in a range of about 6.5 to 6.8. After that, the fermentation of obtained seed culture is performed for 48 hours at about 50° C. under a microaerobic condition and shaking speed around 250 rpm.

### Example 4

Carbon source is 25 ml of liquefied tapioca starch with a concentration of 300 g/L by using calcium carbonate (Ca(CO)₃) to control pH to be in a range of about 6.5 to 6.8. After that, the fermentation of obtained seed culture is performed for 48 hours at about 50° C. under a microaerobic condition and shaking speed around 250 rpm.

The liquefied tapioca starch is obtained from adding alpha-amylase enzyme into tapioca starch solution at a temperature around 100° C. for about 1.5 hours with pH controlled to be around 5.8.

### Example 5

Carbon source is 25 ml of tapioca starch obtained from the method as described in example 4. Calcium carbonate (Ca(CO)₃) is used to control pH to be in a range of about 6.5 to 6.8. After that, the fermentation of obtained seed culture is performed for 48 hours at about 50° C. under a microaerobic condition and shaking speed around 250 rpm. 200 μL of glucoamylase enzyme with a concentration of 37 g/L is added during the fermentation after 2 and 24 hours.

At the end of fermentation, the products are centrifuged at 10,000 rpm for about 5 minutes. The obtained products are analyzed for remaining glucose, an amount of lactic acid produced and optical purity of lactic acid. The results are shown in Table 3.

As seen from Table 3, Bacillus aerolacticus BC-001 could produce lactic acid from various carbon sources including monosaccharide, disaccharide and liquefied starch, provide L-lactic acid with high optical purity that is more than 99%, and high productivity of lactic acid.

The liquefied tapioca starch with the addition of glucoamylase enzyme during fermentation yields the highest productivity of lactic acid.

TABLE 3
Lactic acid production of Bacillus aerolacticus BC-001 from various carbon sources

| | | Lactic acid | | | | |
|---|---|---|---|---|---|---|
| | | Final | | | Optical purity of | Remaining |
| | Type of carbon | concentration | Yield | Productivity | L-lactic acid | glucose |
| Example | sources (g/L) | (g/L) | (g/g) | (g/L/hr) | (% ee) | (g/L) |
| 1 | Glucose (120) | 174.72 | 0.96 | 3.7 | 100.0 | 34.2 |
| 2 | Glucose (100 + 50) | 181.87 | 0.96 | 3.8 | 100.0 | 3.9 |
| 3 | Sucrose (150) | 173.94 | 0.89 | 3.6 | 100.0 | ND |
| 4 | Liquefied tapioca | 21.58 | 0.10 | 0.5 | 100.0 | ND |
| | starch (150) | | | | | |
| 5 | Liquefied tapioca starch | 195.13 | 0.98 | 4.2 | 99.7 | ND |
| | with the addition of | | | | | |
| | glucoamylase enzyme | | | | | |
| | during fermentation (150) | | | | | |

Remarks:
For sucrose, remaining glucose is reported as remaining sucrose
“ND” refers that the remaining glucose cannot be detected.

### BEST MODE OF THE INVENTION

Best mode of the invention is as disclosed in the detailed description.

1. An isolated Bacillus aerolacticus BC-001 species (NITE BP-01943) as deposited to microorganisms depositary institute, which produces L-lactic acid or its salts.

2. A method for producing L-lactic acid or its salts, comprising the following steps:
(1) cultivating the Bacillus aerolacticus species according to claim 1 to obtain a seed culture; and
(2) fermenting the seed culture obtained from step (1) in a carbon source.

3. The method for producing L-lactic acid or its salts according to claim 2, wherein the cultivation in step (1) is performed for a period of 2 to 10 hours.

4. The method for producing L-lactic acid or its salts according to claim 3, wherein the cultivation in step (1) is performed for the period of 3 to 5 hours.

5. The method for producing L-lactic acid or its salts according to claim 4, wherein the cultivation in step (1) is performed for 5 hours.

6. The method for producing L-lactic acid or its salts according to claim 2, wherein a concentration of the Bacillus aerolacticus species in the cultivation step is in a range of 0.5 to 5% by volume.

7. The method for producing L-lactic acid or its salts according to claim 6, wherein the concentration of the Bacillus aerolacticus species in the cultivation step is in the range of 0.5 to 2% by volume.

8. The method for producing L-lactic acid or its salts according to claim 7, wherein the concentration of the Bacillus aerolacticus species in the cultivation step is 1% by volume.

9. The method for producing L-lactic acid or its salts according to claim 2, wherein the fermentation of seed culture in step (2) is performed at a temperature in a range of 45 to 60° C.

10. The method for producing L-lactic acid or its salts according to claim 9, wherein the fermentation of seed culture in step (2) is performed at the temperature of 50° C.

11. The method for producing L-lactic acid or its salts according to claim 2, wherein the fermentation of seed culture in step (2) is performed under a microaerobic condition.

12. The method for producing L-lactic acid or its salts according to claim 2, wherein the carbon source is selected from fermentable sugar, starch, or a mixture thereof.

13. The method for producing L-lactic acid or its salts according to claim 12, wherein the fermentable sugar is monosaccharide selected from glucose, fructose, galactose, or a mixture thereof.

14. The method for producing L-lactic acid or its salts according to claim 12, wherein the fermentable sugar is disaccharide selected from sucrose, lactose, maltose, cellobiose, or a mixture thereof.

15. The method for producing L-lactic acid or its salts according to claim 12, wherein the fermentable sugar is trisaccharide selected from raffinose, isomaltotriose, maltotriose, nigerotriose, kestose, or a mixture thereof.

16. The method for producing L-lactic acid or its salts according to claim 12, wherein the fermentable sugar is selected from glucose, sucrose, or a mixture thereof.

17. The method for producing L-lactic acid or its salts according to claim 12, wherein the starch is liquefied starch selected from tapioca starch, corn starch, wheat starch, or potato starch that have been contacted to amylase enzyme.

18. The method for producing L-lactic acid or its salts according to claim 2, wherein the concentration of carbon source in the fermentation of seed culture is in a range of 50 to 200 g/L.

19. The method for producing L-lactic acid or its salts according to claim 2, wherein the fermentation of seed culture in step (2) further comprises a step of adding glucoamylase enzyme during the fermentation of seed culture.

20. The method for producing L-lactic acid or its salts according to claim 2, wherein L-lactic acid or its salts have an optical purity more than 95%.

21. The method for producing L-lactic acid or its salts according to claim 20, wherein L-lactic acid or its salts have the optical purity more than 99%.


SEQUENCE LISTING (1 sequence)

SEQ ID NO: 1 - DNA, 1458 bp, Bacillus aerolacticus
cctaatacatgcaagtcgagcggaccttttaaaagcttgcttttgaaaggtcagcggcggacgggtgagtaacacgtgggtaacctgcctgtgagactgggataactccgggaaaccggggctaataccggataggattttcctccgcatggaggaaaatgaaaagatggcttttgctatcacttacagatggacccgcggcgcattagctggttggtgaggtaacggctcaccaaggcgacgatgcgtagccgacctgagagggtgatcggccacattgggactgagacacggcccaaactcctacgggaggcagcagtagggaatcttccgcaatggacgaaagtctgacggagcaacgccgcgtgagtgaagaaggttttcggatcgtaaaactctgttgtcagggaagaacaagtgccgttcgaacagggcggcaccttgacggtacctgaccagaaagccacggctaactacgtgccagcagccgcggtaatacgtaggtggcaagcgttgtccggaattattgggcgtaaagcgcgcgcaggcggcttcttaagtctgatgtgaaatcttgcggctcaaccgcaagcggtcattggaaactgggaggcttgagtgcagaagaggagagtggaattccacgtgtagcggtgaaatgctagagatgtggaggaacaccagtggcgaaggcgactctctggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctctgacctccctggagacagggacttccccttcgggggacagagtgacaggtggtgcatggttgtcgtcagctcgtgtcgtgagatgttgggttaagtcccgcaacgagcgcaacccttgaccttagttgccagcattcagttgggcactctaaggtgactgccggtgacaaaccggaggaaggtggggatgacgtcaaatcatcatgccccttatgacctgggctacacacgtgctacaatggatggtacaaagggcagcgagaccgcgaggttaagccaatcccataaaaccattctcagttcggattgcaggctgcaacccgcctgcatgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattgggggtgaa